(19) United States
(12) Patent Application Publication
(10) Pub. No.: US 2006/0035871 A1
(43) Pub. Date: Feb. 16, 2006

(54) Pulverulent phytosterol formulations

(75) Inventors: Helmut Auweter, Limburgerhof (DE); Heribert Bohn, Wattenheim (DE); Oliver Hasselwander, Landau (DE); Frank Runge, Friedelsheim (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: BASF Aktiengesellschaft, Ludwigshafen (DE)

(21) Appl. No.: 10/534,543

(22) PCT Filed: Nov. 11, 2003
(86) PCT No.: PCT/EP03/12557

§ 371 Date: May 10, 2005

(30) Foreign Application Priority Data
Nov. 13, 2002 (DE) 102 53 111.0

(51) Int. Cl. A61K 31/56

(52) U.S. Cl. 514/169; 424/442

(57) ABSTRACT

A pulverulent phytosterol formulation comprising at least one phytosterol having a mean particle size ranging from 0.01 to 100 μm, a process for producing the pulverulent phytosterol formulation, and a food supplement, an animal feed, food or pharmaceutical or cosmetic preparation comprising the phytosterol formulation.

The invention relates to pulverulent phytosterol formulations, processes for their production and their use in food supplements, in foods and animal feeds and also in pharmaceutical and cosmetic preparations.

Phytosterols are sterols which are isolated from plants and yeasts. The most important members of this class of compounds are, for example, stigmasterol, campesterol and β-sitosterol and also hydrogenated derivatives such as campestanol and β-sitostanol. Phytosterols are structurally similar to cholesterol. Since, for example, β-sitosterol inhibits the absorption of cholesterol, it is used as a lipid reducer for the prophylaxis of arteriosclerosis and hyperlipaemia.

To lower the cholesterol level, phytosterols are frequently being used as additives in dietetic foods, for example margarines.

Phytosterols are insoluble in water, while only a low solubility has been found in fats and oils. This limited solubility frequently complicates the employability of phytosterols in the production of food preparations and cosmetic products. Inadequate activities on the one hand and poor dispersibility in cosmetics and food preparations on the other frequently result from the poor solubilities of phytosterols.

Various processes for producing phytosterol-containing formulations are already known. Thus EP-A-0 289 636 describes solubilized phytosterols in an aqueous solution of polyhydroxy compounds or sucrose esters of fatty acids.

Other liquid preparations of phytosterols together with solubilizers are disclosed in U.S. Pat. No. 3,865,939 and U.S. Pat. No. 5,244,887.

EP-A-1 197 153 describes aqueous dispersions or suspensions of phytosterols in the presence of non-sterol-like emulsifiers and their use in foods, for example in bread spreads.

WO 01/37681 relates to aqueous phytosterol-containing compositions obtainable by homogenizing phytosterols in water in the presence of a water-soluble protein, for example in the presence of casein, and water-dispersible powders produced therefrom.

It is an object of the present invention to provide phytosterol-containing formulations which can be incorporated into not only aqueous preparations, but also oily preparations.

We have found that this object is achieved according to the invention by pulverulent phytosterol formulations comprising at least one phytosterol having a mean particle size in the range from 0.01 to 100 μm, preferably in the range from 0.01 to 10 μm, particularly preferably in the range from 0.01 to 2 μm, very particularly preferably in the range from 0.05 to 1 μm.

For the purposes of the present invention phytosterols are preferably the three compounds stigmasterol, campesterol and β-sitosterol, and also their hydrogenated derivatives stigmastanol, campestanol and β-sitostanol. Particular preference is given to the phytosterol mixtures produced from soybean oil by distillation, which consist essentially of stigmasterol, campesterol and β-sitosterol.

A typical mixture of these three phytosterols which is produced from vegetable oils consists of approximately from 40 to 58% by weight of β-sitosterol, from 20 to 30% by weight of campesterol and from 14 to 22% by weight of stigmasterol.

The inventive phytosterol formulations are, inter alia, also distinguished in that at least one phytosterol is present in partially amorphous form.

The degree of crystallinity of the phytosterols in the inventive formulations may be determined, for example, by X-ray diffraction measurements and is generally in the range of less than 80%, preferably in the range from 30 to 80%, particularly preferably in the range from 50 to 80%.

In a further preferred embodiment of the phytosterol formulations, the phytosterol is embedded in a protective colloid matrix.

Suitable protective colloids are both electrically charged polymers (polyelectrolytes) and neutral polymers. Typical examples are, inter alia, gelatin, such as cattle, swine or fish gelatin, starch, modified starch such as octenylsuccinate starch, dextrin, plant proteins such as soybean proteins, which may be hydrolyzed, pectin, guar gum, xanthan, gum arabic, casein, sodium caseinate, lignosulfonate, or mixtures thereof. However, methylcellulose, carboxymethylcellulose, hydroxypropylcellulose, hydroxypropylmethylcellulose, sheet shellac and alginates can also be used. Other suitable compounds are homopolymers and copolymers based on neutral, cationic or anionic monomers, for example ethylene oxide, propylene oxide, acrylic acid, maleic anhydride, lactic acid, N-vinylpyrrolidone, vinyl acetate, α- and β-aspartic acid. For further details, reference is made to R. A. Morton, Fat Soluble Vitamins, Intern. Encyclopedia of Food and Nutrition, Vol. 9, Pergamon Press 1970, pp. 128-131.

Preferred protective colloids are compounds selected from the group consisting of gelatin such as cattle, swine and fish gelatin, plant proteins, pectin, casein, sodium caseinate, gum arabic and modified starch. Particularly preferred protective colloids are pectin, casein, sodium caseinate, gum arabic, modified starch and/or fish gelatin.

The phytosterol content in the inventive formulations is in the range from 0.1 to 80% by weight, preferably from 1 to 50% by weight, particularly preferably from 3 to 35% by weight, very particularly preferably in the range from 5 to 25% by weight, the percentages by weight being based on the dry matter of the powder.

The amount of protective colloids used is in the range from 0.1 to 80% by weight, preferably from 5 to 70% by weight, particularly preferably in the range from 10 to 60% by weight. The percentages by weight are based on the dry matter of the phytosterol formulation.

In addition the phytosterol formulations can further comprise one or more plasticizers to increase the mechanical stability of the powders. Suitable plasticizers are, for example, sugars and sugar alcohols such as sucrose, glucose, lactose, invert sugar, sorbitol, mannitol, xylitol or glycerol. The plasticizers can be present in amounts of from 0.1 to 70% by weight, preferably from 10 to 60% by weight, particularly preferably from 20 to 50% by weight, based on the dry matter of the phytosterol formulations.

In addition, the formulations can comprise one or more low-molecular-weight surface-active compounds (emulsifiers) in a concentration of from 0.01 to 70% by weight, preferably from 0.1 to 50% by weight, particularly preferably from 0.5 to 20% by weight, based on the dry matter of the phytosterol formulations. Those which are suitable are primarily amphiphilic compounds or mixtures of such compounds. In principle, those which can be used are all surfactants for food or feed use and are pharmacologically and dermatologically safe and have an HLB value of from 5 to 20. Corresponding surface-active substances which can be used are, for example: esters of long-chain fatty acids with ascorbic acid, mono- and diglycerides of fatty acids and their ethoxylation products, esters of fatty acid monoglycerides of acetic acid, citric acid, lactic acid or diacetyltartaric acid, polyglycerol esters of fatty acids, for example the monostearate of triglycerol, sorbitan fatty acid esters, propylene glycol fatty acid esters and lecithin. Preferably, ascorbyl palmitate is used.

In addition, the formulations can further comprise one or more low-molecular-weight stabilizers such as antioxidants and/or preservatives. Suitable antioxidants or preservatives are, for example, α-tocopherol, ascorbic acid, tert-butylated hydroxytoluene, tert-butylated hydroxyanisole, lecithin, ethoxyquine, methylparaben, propylparaben, sorbic acid or sodium benzoate. The antioxidants or preservatives can be present in amounts of from 0.01 to 50% by weight, preferably from 0.1 to 30% by weight, particularly preferably from 0.5 to 20% by weight, very particularly preferably from 1 to 10% by weight, based on the dry matter of the phytosterol formulations.

In addition to the phytosterols, the inventive formulations can additionally further comprise carotinoids and vitamins. Examples of carotinoids are, inter alia, β-carotene, bixin, zeaxanthin, cryptoxanthin, citranaxanthin, canthaxanthin, β-apo-4-carotenal, β-apo-8-carotenal, β-apo-8-carotenic acid esters, astaxanthin, lycopene or lutein, individually or as a mixture.

Of the vitamins, preference is given to fat-soluble vitamins such as vitamin E, vitamin E derivatives, for example tocopheryl acetate or tocopheryl palmitate, and also the K vitamins, vitamin A and derivatives, for example vitamin A acetate, vitamin A propionate or vitamin A palmitate, vitamin D₂ and vitamin D₃ and mixtures. The term vitamin E, for the purposes of the present invention, means natural or synthetic α-, β-, γ- or δ-tocopherol, preferably natural or synthetic α-tocopherol, and also tocotrienol.

The inventive phytosterol formulations are distinguished, inter alia, in that they are readily dispersible not only in oily systems, but also in aqueous systems, for example in beverages.

The invention also relates to a process for producing the above-described pulverulent phytosterol formulations, which comprises
a₁) dissolving one or more phytosterols in a water-miscible organic solvent or in a mixture of water and a water-miscible organic solvent, or
a₂) dissolving one or more phytosterols in a water-immiscible organic solvent and
b) mixing the solution obtained as in a₁) or a₂) with an aqueous molecular dispersion or colloidal dispersion of a protective colloid, the hydrophobic phase of the phytosterol being formed as disperse phase, and
c) to produce a dry powder, freeing the resulting dispersion from the solvent and the water and drying it in the presence or absence of a coating material.

Depending on the type of solvents used, the disperse phase in step b) can be solid nanoparticles (suspension) or nanodroplets (emulsion).

The water-miscible solvents used in step a₁) are primarily water-miscible, thermally stable, volatile solvents containing only carbon, hydrogen and oxygen, such as alcohols, ethers, esters, ketones and acetals. Expediently, such solvents are used which are water-miscible up to 10%, have a boiling point below 200° C. and/or have fewer than 10 carbons. Particular preference is given to methanol, ethanol, n-propanol, isopropanol, 1,2-butanediol 1-methyl ether, 1,2-propanediol 1-n-propyl ether, tetrahydrofuran and/or acetone, very particular preference is given to n-propanol, isopropanol and/or acetone.

For the purposes of the present invention, “a water-immiscible organic solvent” is an organic solvent having a water solubility at atmospheric pressure of less than 10%. Possible solvents which can be used here are, inter alia, halogenated aliphatic hydrocarbons, for example methylene chloride, chloroform and carbon tetrachloride, carboxylic esters such as dimethyl carbonate, diethyl carbonate, propylene carbonate, ethyl formate, methyl, ethyl or isopropyl acetate, and ethers such as methyl tert-butyl ether. Preferred water-immiscible organic solvents are the following compounds selected from the group consisting of dimethyl carbonate, propylene carbonate, ethyl formate, ethyl acetate, isopropyl acetate and methyl tert-butyl ether.

Protective colloids which are used in the process step b) are the compounds already mentioned at the outset.

In some circumstances it can also be advantageous to add, additionally to the solvent phase, a physiologically acceptable oil, for example sesame oil, corn germ oil, cottonseed oil, soybean oil or peanut oil, and also esters of medium-chain vegetable fatty acids at a concentration of from 0 to 500% by weight, preferably from 10 to 300% by weight, particularly preferably from 20 to 100% by weight, based on the phytosterol(s), which is then precipitated out in an extremely finely divided form together with the active compounds and said additives on mixing with the aqueous phase.

In a preferred embodiment of the inventive process
a) one or more phytosterols is/are dissolved in a water-miscible organic solvent, or a mixture of water and a water-miscible organic solvent, at temperatures in the range from 50° C. to 240° C., preferably in the range from 100° C. to 200° C., particularly preferably from 140° C. to 180° C.,
b) the resultant solution is mixed with an aqueous molecular dispersion or colloidal dispersion of a protective colloid selected from the group consisting of pectin, casein, caseinate, gum arabic, modified starch and fish gelatin, a mixture temperature of from about 35° C. to 80° C. being established and
c) the resultant dispersion is converted into a dry powder.

Very particularly preferably, this is in this case a process for preparing dry powders of a mixture of stigmasterol, campesterol and β-sitosterol.

Since the effect of high temperatures can, under some circumstances, decrease the desired content of phytosterols. The phytosterol(s) is(are) dissolved as quickly as possible, for example in the range of seconds, for example in from 0.1 to 10 seconds, particularly preferably in less than 1 second. For rapid production of the molecular dispersion, the use of evaluated pressure, for example in the range from 20 bar to 80 bar, preferably from 30 to 60 bar, can be advantageous.

The resultant molecular dispersion is then admixed directly with the possibly cooled aqueous molecular dispersion or colloidal dispersion of the protective colloid in such a manner that a mixture temperature of from about 35° C. to 80° C. is established.

The solvent component is transferred to the aqueous phase and the hydrophobic phase of the phytosterol/phytosterols results as disperse phase.

The mean particle size of the nanoparticulate particles in the aqueous dispersion is, depending on the type of formulation method, in the range from 0.01 to 100 μm, preferably in the range from 0.01 to 10 μm, particularly preferably in the range from 0.01 to 2 μm, very particularly preferably in the range from 0.05 to 1 μm.

At this point, reference is made to EP-B-0 065 193 with respect to a more detailed description of process and apparatus for the abovementioned dispersion.

The conversion into a dry powder can be performed here, inter alia, by spray-drying, spray-chilling, freeze-drying or drying in a fluidized bed, in the presence of absence of a coating material. Suitable coating materials are, inter alia, corn starch, silica or else tricalcium phosphate.

The invention also relates to a process for producing the abovementioned pulverulent phytosterol formulations, which comprises grinding at least one phytosterol in an aqueous medium in the presence of a protective colloid and drying the resultant phytosterol suspension to produce a dry powder.

The grinding can be performed in a manner known per se, for example using a ball mill. Depending on the type of mill used, grinding is carried out until the particles have a mean particle size of from 0.01 to 100 μm, preferably from 0.2 to 50 μm, particularly preferably from 0.2 to 20 μm, very particularly preferably from 0.2 to 5 μm, in particular from 0.2 to 0.8 μm.

Further details on grinding and the apparatuses used therefor may be found, inter alia, in Ullmann's Encyclopedia of Industrial Chemistry, Sixth Edition, 1999, Electronic Release, Size Reduction, Chapter 3.6.: Wet Grinding, and also in EP-A-0 498 824.

In a further variant of the abovementioned grinding process, the phytosterol suspension, after the grinding, is heated to a sufficiently high temperature to cause complete or partial melting of the phytosterols and this melt is cooled again before being converted into a dry powder. Preferably, here, the phytosterol suspension, after the grinding, is kept at a temperature of from 150 to 200° C. for a period of from 0.05 to 200 seconds, preferably from 0.2 to 100 seconds, and is cooled to a temperature of from 20 to 80° C. before conversion into a dry powder.

Depending on the drying method, the phytosterol-containing dry powders have a mean particle size of from 100 to 1000 μm, preferably from 200 to 800 μm, particularly preferably from 250 to 600 μm. These powders are agglomerates (secondary particles) of the primary particles already described at the outset having a mean particle size in the range from 0.01 to 100 μm.

The particle size, both of the primary particles and of the secondary particles, is determined here using known methods of measurement, for example via Fraunhofer diffraction and, in the case of particles smaller than 5 μm, using dynamic light scattering.

The inventive dry powders may be redispersed again without problems in aqueous systems to achieve a uniform fine distribution of the active compound in the particle size range from 0.01 to 1 μm.

The inventive phytosterol formulations are suitable, inter alia, as additive for food preparations and animal feeds, as compositions for producing pharmaceutical and cosmetic preparations and also for producing food supplement preparations in the human and animal sectors.

A typical field of application in the food sector is, for example, use in beverages, milk products such as cheese, yoghurt, flavored milk drinks or dairy ice cream, and also salad dressings, sauces and mayonnaises, but also in sausage products and confectionery.

Preferably, the suspensions may be used in animal nutrition as feed additives, in particular for application or spraying onto feed pellets.

The use as feed additive takes place in particular in the form of liquid preparations in which the inventive pulverulent phytosterol formulations are dispersed in an oil.

Oils which can be used are generally all physiologically compatible oils, both of vegetable and animal origin, in particular those oils which are liquid at 20° C. or which form the liquid phase in the suspension at 20° C. alone or together with other oils. Those which may preferably be mentioned in this context are sunflower oil, palm oil, sesame oil, corn germ oil, cottonseed oil, soybean oil or peanut oil, esters of medium-chain triglycerides and in addition fish oils, for example mackerel oil, sprat oil or salmon oil. Those which are particularly preferred for animal nutrition are fish oils, corn germ oil, sunflower oil and peanut oil.

These liquid preparations can be applied, for example, by direct spraying onto animal feed pellets in what is called a post-pelleting application.

A preferred embodiment of the spraying process is that, for example, the feed pellets are charged with the oily suspension at reduced pressure.

Examples of this may be found, inter alia, in GB-A-2 232 573 and in EP-A-0 556 883.

Typical fields of use in the food sector are, for example, the vitaminization of beverages, milk products such as yoghurt, flavored milk drinks or dairy ice cream and of pudding powders, egg products, baking mixes and confectionery.

In the cosmetics sector, the oily suspensions can be used, for example, for vitamin-containing body care products, for example in the form of a cream, a lotion, as lipsticks or makeup.

In the cosmetics sector, the inventive phytosterol formulations can be used, for example, as emollient or else as active compound in skincare products.

The invention also relates to food supplements, animal feeds, foods and pharmaceutical and cosmetic preparations comprising the above-described phytosterol formulations.

For the purposes of the present invention, food supplement preparations are pharmaceutical preparations which comprise the inventive phytosterol formulation, inter alia tablets, dragees and hard and soft gelatin capsules.

For the purposes of the present invention, foods are, for example, beverages, milk products such as cheese, yoghurt, flavored milk drinks or dairy ice cream and also salad dressings, sauces or mayonnaises, confectionery and sausage products which comprise the above-described phytosterol formulations.

Cosmetics preparations which can comprise the inventive phytosterol formulations are, for example, preparations which can be applied topically, in particular skincare products and decorative body-care products, such as lipsticks, face makeup in the form of a cream, a lotion, a powder or else as rouge.

The pharmaceutical preparations are suitable for prophylaxis or therapy of an excessive cholesterol level.

In the examples below the production of the inventive phytosterol formulations is described in more detail. Details of the equipment set-up used in the examples may be found in EP-B-0 065 193.

### EXAMPLE 1

Dry Phytosterol Powder Containing Sodium Caseinate

21 g of phytosterol (from ADM, USA) and 2.1 g of ascorbyl palmitate were dissolved in 360 g of acetone at room temperature in a receptacle. In a second receptacle, 35 g of Na caseinate and 35 g of sucrose were dissolved in 4000 g of demineralized water at 70° C. The solvent phase which was set to 86.8° C. was then continuously mixed at a pumping rate of 0.92 kg/h with the aqueous phase at room temperature and a pumping rate of 30.3 kg/h. The resultant active compound dispersion was freed of acetone on a rotary evaporator at 65° C. and a pressure of 200 mbar and concentrated to a solids content of 11.5% by weight. The resultant active compound particles had a particle size of 203 nm.

This dispersion was then spray dried on a laboratory spraying tower. The phytosterol content in the resultant dry powder was 26% by weight. The dry powder is dispersible in water, and after redispersion gave a particle size of 1.08 μm.

### EXAMPLE 2

Dry Phytosterol Powder Containing Modified Starch

21 g of phytosterol (from ADM, USA) and 2.1 g of ascorbyl palmitate were dissolved in 360 g of acetone at room temperature in a receptacle. In a second receptacle, 35 g of modified starch (Emcap 12633, from Cerestar, Krefeld) and 35 g of sucrose were dissolved in 4000 g of demineralized water at 70° C. The solvent phase which was set to 94.9° C. was then, at a pumping rate of 2.61 kg/h, continuously mixed with the aqueous phase at room temperature and a pumping rate of 30.0 kg/h. The resultant active compound dispersion was freed of acetone on a rotary evaporator at 65° C. and a pressure of 200 mbar and concentrated to a solids content of 9.1% by weight. The resultant active compound particles had a particle size of 264 nm.

This dispersion was then spray dried on a laboratory spraying tower. The phytosterol content in the resultant dry powder was 20.7% by weight. The dry powder is dispersible in water and, after redispersion, gave a particle size of 2.3 μm.

### EXAMPLE 3

Dry Phytosterol Powder Containing Modified Starch

40 g of phytosterol (from ADM, USA), 6 g of ascorbyl palmitate and 40 g of modified starch (Capsul MKH, from National Starch, Hamburg) were suspended at room temperature in 400 g of demineralized water. The pH was then adjusted to 7.1 using 1 M NaOH. This suspension, together with 2000 g of ceramic balls (zirconium oxide, Toray) of diameter 1 mm, was then placed in a 1000 ml glass flask. The suspension was then dispersed in this glass flask for 8 hours on a dispersion unit (Red Devil). The active compound particles then had a size of 585 nm.

After separating off the milling media, 344 g of dispersion were obtained. 28.3 g of sucrose were dissolved in this. This dispersion was then spray dried on a laboratory spraying tower. The phytosterol content in the resultant dry powder was 19.2% by weight. Dry powder is dispersible in water and, after redispersion, gave a particle size of 1.2 μm.

1. A pulverulent phytosterol formulation comprising at least one phytosterol having a mean particle size of from 0.01 to 100 μm.

2. The phytosterol formulation as claimed in claim 1, wherein at least one phytosterol is present in partially amorphous form.

3. The phytosterol formulation as claimed in claim 1, wherein the phytosterol is embedded in a protective colloid matrix.

4. The phytosterol formulation as claimed in claim 1, comprising from 0.1 to 80% by weight of one or more phytosterols, with the percentages by weight being based on the dry matter of the powder.

5. The phytosterol formulation as claimed in claim 4, comprising from 5 to 70% by weight of one or more protective colloids.

6. The phytosterol formulation as claimed in claim 1, additionally comprising from 0.1 to 70% by weight of one or more plasticizers.

7. The phytosterol formulation as claimed in claim 1, additionally comprising from 0.01 to 70% by weight of one or more emulsifiers.

8. The phytosterol formulation as claimed in claim 1, additionally comprising from 0.01 to 50% by weight of one or more antioxidants and/or preservatives.

9. The phytosterol formulation as claimed in claim 1, which is water-dispersible.

10. A process for producing a pulverulent phytosterol formulation defined as claimed in claim 1, which comprises:
a₁) dissolving one or more phytosterols in a water-miscible organic solvent or in a mixture of water and a water-miscible organic solvent, or
a2) dissolving one or more phytosterols in a water-immiscible organic solvent; and
b) mixing the solution obtained as in a1) or a2) with an aqueous molecular dispersion or colloidal dispersion of a protective colloid, the hydrophobic phase of the phytosterol being formed as disperse a dispersed phase,
c) to produce a dry powder, freeing the resulting dispersion from the solvent and the water and drying it in the presence or absence of a coating material.

11. A The process as claimed in claim 10, wherein
a) one or more phytosterols is/are dissolved in a water-miscible organic solvent, or a mixture of water and a water-miscible organic solvent, at temperatures in the range from 50° C. to 240° C.;
b) the resultant solution is mixed with an aqueous molecular dispersion or colloidal dispersion of a protective colloid selected from the group consisting of pectin, casein, caseinate, gum arabic, modified starch and fish gelatin, a mixture temperature of from about 35° C. to 80° C. being established; and
c) the resultant dispersion is converted into a dry powder.

12. A process for producing a pulverulent phytosterol formulation defined as claimed in claim 1, which comprises grinding at least one phytosterol in an aqueous medium in the presence of a protective colloid and drying the resultant phytosterol suspension to produce a dry powder.

13. The process as claimed in claim 12, wherein the phytosterol suspension, after the grinding, is heated to a sufficiently high temperature to cause complete or partial melting of the phytosterols and this melt is cooled again before being converted into a dry powder.

14. The process as claimed in claim 13, wherein the phytosterol suspension, after the grinding, is kept at a temperature of from 150 to 200° C. for a period of from 0.05 to 200 seconds and is cooled to a temperature of from 20 to 80° C. before conversion into a dry powder.

15. (canceled)

16. A food supplement, animal feed, food or pharmaceutical or cosmetic preparation comprising a phytosterol formulation defined as claimed in claim 1.
